(19) United States
(12) Patent Application Publication
(10) Pub. No.: US 2016/0143738 A1
(43) Pub. Date: May 26, 2016

(54) Biological Constructs for Treating Damaged Organs and Tissue

(71) Applicant: CorMatrix Cardiovascular, Inc., Roswell, GA (US)

(72) Inventor: Robert G. Matheny, Norcross, GA (US)

(21) Appl. No.: 14/957,995

(22) Filed: Dec. 3, 2015

Related U.S. Application Data
(63) Continuation-in-part of application No. 14/554,730, filed on Nov. 26, 2014.

(51) Int. Cl. A61F 2/24; A61K 38/18; A61F 2/00; A61K 31/44

(52) U.S. Cl. CPC A61F 2/2481; A61K 31/44; A61K 38/1841; A61F 2/0077; A61F 2002/0086; A61F 2210/0004; A61F 2210/0057; A61F 2230/0069; A61F 2230/0067; A61F 2250/0067

(57) ABSTRACT

Biological constructs that can be engineered into a variety of shapes and employed to treat, augment and/or support damaged or diseased mammalian organs and/or tissue related thereto. The shapes include jackets and bands that are configured to encase a preselected region of a mammalian organ.

### CROSS-REFERENCES TO RELATED APPLICATIONS

This application is a continuation-in-part of U.S. application Ser. No. 14/554,730, filed on Nov. 26, 2014.

### FIELD OF THE INVENTION

The present invention relates to implantable biological constructs for treating damaged or diseased tissue. More particularly, the present invention relates to non-antigenic, resilient, biocompatible biological constructs that can be engineered into a variety of shapes and used to treat, augment and/or support damaged or diseased mammalian organs and/or tissue related thereto.

### BACKGROUND OF THE INVENTION

As is well known in the art, implantable biological constructs, such as vascular prostheses, are often employed to treat damaged or diseased biological tissue. However, despite the growing sophistication of medical technology, the use of biological constructs to treat damaged biological tissue remains a frequent and serious problem in health care. The problem is often associated with the materials employed to form the biological constructs.

As is also well known in the art, the optimal biological construct material should be chemically inert, non-carcinogenic, capable of resisting mechanical stress, capable of being fabricated in the form required, and sterilizable. Further, the material should not excite an inflammatory reaction, induce a state of allergy or hypersensitivity, or, in some cases, promote visceral adhesions.

Various materials have thus been employed to form biological constructs in an attempt to address one or more of the aforementioned optimal characteristics, including tantalum gauze, stainless mesh, nitinol, Dacron®, Orlon®, Fortisan®, nylon, knitted polypropylene (e.g., Marlex®), microporous expanded-polytetrafluoroethylene (e.g., Gore-Tex®), Dacron reinforced silicone rubber (e.g., Silastlc®), polyglactin 910 (e.g., Vicryl®), polyester (e.g., Mersilene®), polyglycolic acid (e.g., Dexon®), processed sheep dermal collagen, crosslinked bovine pericardium (e.g., Peri-Guard®), and preserved human dura (e.g., Lyodura®).

As discussed in detail below, although some of the noted materials address one or more of the aforementioned optimal characteristics, there still remains several, and in some instances major, drawbacks associated with the use of such materials to form implantable biological constructs.

For example, although conventional metal constructs are inert and generally resistant to infection, metal constructs are often prone to fragmentation, which can, and in many instances will, occur after the first year of administration.

Marlex®, i.e. polypropylene, which is widely used to form biological constructs for abdominal wall replacement and reinforcement during hernia repairs, can, and in many instances, will induce scar contracture. Marlex® constructs are also prone to distortion and often separate from surrounding normal tissue.

Gore-Tex®, i.e. polytetrafluoroethylene, is also often employed to form biological constructs. Although Gore-Tex® is deemed a highly chemically inert graft material, when a Gore-Tex® construct is disposed proximate a contaminated wound it does not allow for any macromolecular drainage, which limits treatment of infections.

Collagen is another material that is commonly employed to form biological constructs. Collagen constructs are, however, typically crosslinked with agents, such as glutaraldehyde, formaldehyde and photo-iodide, to enhance mechanical strength and decrease the degradation rate of the collagen.

A major disadvantage of crosslinking collagen is, however, that it enhances the antigenicity of the material by linking the antigenic epitopes, rendering them either inaccessible to phagocytosis or unrecognizable by the immune system. Crosslinking also changes the microarchitecture of the collagen altering normal cell recognition and interaction and essentially creating a synthetic construct.

Extracellular matrix (ECM) derived from mammalian tissue has also recently garnered considerable success as a biological construct material. Illustrative are the biological constructs disclosed in Applicant's U.S. Pat. Nos. 8,758,448 and 9,066,993, and Co-Pending U.S. application Ser. No. 13/328,287.

Although ECM based biological constructs have garnered considerable success by addressing many of the aforementioned optimal characteristics, efforts continue to develop improved biological constructs that can successfully be formed in various shapes and employed to facilitate the repair of damaged mammalian organs and/or tissue associated therewith.

It is therefore an object of the present invention to provide improved biological constructs that address many of the aforementioned optimal characteristics; particularly, provide a minimal risk of inciting an inflammatory reaction or state of hypersensitivity, as well as being capable of resisting mechanical stress and being formed in a variety of desired shapes.

It is another object of the present invention to provide biological constructs that induce modulated healing of damaged mammalian organs and/or tissue associated therewith, including modulated inflammation of the damaged tissue and/or neovascularization, host tissue proliferation, bioremodeling, and regeneration of tissue and associated structures with site-specific structural and functional properties, when disposed proximate thereto.

It is another object of the present invention to provide biological constructs that induce adaptive regeneration of damaged mammalian organs, including stress-induced hypertrophy, when disposed proximate thereto.

It is yet another object of the invention to provide means for treating and/or supporting damaged or diseased mammalian organs and/or tissue related thereto by employing biological constructs that induce modulated healing and, in some instances, adaptive regeneration.

### SUMMARY OF THE INVENTION

The present invention is directed to biocompatible biological constructs that can be engineered into a variety of shapes and employed to treat and/or support damaged or diseased mammalian organs and/or tissue related thereto, and methods for employing same to treat and/or support damaged or diseased mammalian organs and/or tissue related thereto.

In a preferred embodiment of the invention, the biological constructs are configured to form a jacket or band that is configured to encase a preselected region of a mammalian organ.

In some embodiments of the invention, the biological constructs comprise a mesh fiber member.

In some embodiments of the invention, the biological constructs comprise a substantially solid member or sheet.

In some embodiments of the invention, the comprise an ECM composition comprising at least one ECM derived from a mammalian tissue source selected from the group comprising, without limitation, the small intestine, large intestine, stomach, lung, liver, kidney, pancreas, placenta, amniotic membrane, heart, bladder, prostate, tissue surrounding growing enamel, tissue surrounding growing bone, and any fetal tissue from any mammalian organ. The ECM can also comprise collagen from mammalian sources.

In some embodiments of the invention, the biological constructs comprise an ECM-mimicking polymeric composition comprising poly(glycerol sebacate) (PGS).

In some embodiments of the invention, the biological constructs comprise an ECM-PGS composition.

In some embodiments, the biological constructs comprise a PVA composition comprising polyvinyl alcohol.

In some embodiments of the invention, the biological constructs comprise at least one additional biologically active agent, i.e. an agent that induces or modulates a physiological or biological process, or cellular activity, e.g., induces cell attraction and proliferation, and/or growth and/or regeneration of tissue.

In some embodiments, the biologically active agent comprises a cell selected from the group comprising, without limitation, embryonic stem cells, mesenchymal stem cells, hematopoietic stem cells, bone marrow stern cells, bone marrow-derived progenitor cells, myosatellite progenitor cells, totipotent stem cells, pluripotent stem cells, multipotent stem cells, oligopotent stem cells and unipotent stem cells.

In some embodiments, the biologically active agent comprises a growth factor selected from the group comprising, without limitation, transforming growth factor alpha (TGF-α), transforming growth factor beta (TGF-β), fibroblast growth factor-2 (FGF-2), vascular epithelial growth factor (VEGF), insulin-like growth factor (IGF) and hepatic growth factor (HGF).

In some embodiments, the biologically active agent comprises a protein selected from the group comprising, without limitation, collagen (types I-V), proteoglycans, glycosaminoglycans (GAGs), glycoproteins, cytokines, cell-surface associated proteins, and cell adhesion molecules (CAMs).

In some embodiments of the invention, the biologically active agent is selected from the group comprising RNAs, DNAs and nucleic acid sequence compounds that alter the genetic expression of host cells within the myocardium and epicardium, including, without limitation, progenitor cells, fibroblasts and mesothelial cells. The later may or may not use virally directed genetic coding that function in genome altering sequences or act upon epigenetic areas to activate or deactivate genes.

In some embodiments of the invention, the biological constructs comprise at least one pharmacological agent or composition, i.e. an agent, drug, compound, composition of matter or mixture thereof, including its formulation, which provides some therapeutic, often beneficial, effect.

In some embodiments, the pharmacological agent or composition is selected from the group comprising, without limitation, antibiotic, anti-arrhythmic and anti-viral agents, and anticoagulant and/or antithrombic agents.

In some embodiments of the invention, the biological constructs further comprise at least one coating, which, optionally, can include one of the aforementioned biologically active or pharmacological agents.

In some embodiments, the coating comprises an ECM composition.

In some embodiments, the coating comprises an ECM-mimicking composition.

In some embodiments, the coating comprises an ECM-PGS composition.

In some embodiments, the coating comprises a polymeric composition.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of one embodiment of a strand that is employed to form a mesh biological construct, in accordance with the invention;

FIG. 2 is a perspective sectional view of another embodiment of a strand that is employed to form a mesh biological construct, in accordance with the invention;

FIG. 3 is a front plan view of a fiber construct that is employed to form a mesh biological construct, in accordance with the invention;

FIGS. 4-7 are top plan views of several embodiments of the mesh biological constructs, in accordance with the invention;

FIG. 8 is a graphical illustration reflecting the effect of a statin augmented ECM on MCP-1 mRNA expression over time, in accordance with the invention;

FIG. 9 is a graphical illustration reflecting the effect of a statin augmented ECM on CCR2 mRNA expression over time, in accordance with the invention;

FIG. 10 is a graphical illustration reflecting the effect of a statin augmented ECM on RAC1 mRNA expression over time, in accordance with the invention;

FIG. 11 is a graphical illustration reflecting the effect of a statin augmented ECM on MCP-1 concentration and mRNA expression over time, in accordance with the invention.

FIG. 12 is a depiction of a mammalian heart;

FIG. 13 is a perspective view of one embodiment of a biological construct in the form of an organ encasement jacket, in accordance with the invention;

FIG. 14 is a bottom plan view of another embodiment of a biological construct in the form of an organ encasement band, in accordance with the invention;

FIG. 15 is a depiction of the organ encasement band shown in FIG. 14 pre-positioned proximate a region of a mammalian heart, in accordance with the invention;

FIG. 16A is a perspective view of the organ encasement jacket shown in FIG. 13 positioned on a mammalian heart, in accordance with the invention;

FIG. 16B is a perspective view of the organ encasement band shown in FIGS. 14 and 15 positioned around a region of a mammalian heart, in accordance with the invention;

FIG. 17 is a front plan sectional view of one embodiment of an organ encasement jacket having a coating disposed on the encasement surface, in accordance with the invention;

FIG. 18 is a front plan sectional view of one embodiment of an organ encasement jacket having multiple coatings; a first coating disposed on the encasement surface and a second coating disposed on the exterior surface, in accordance with the invention;

FIG. 19 is a front plan view of one embodiment of a coated organ encasement band, in accordance with the invention;

FIGS. 20-27 are top plan views of several embodiments of biological construct support structures, in accordance with the invention;

FIG. 28 is a front plan sectional view of one embodiment of a reinforced biological construct, i.e. a reinforced organ encasement band, in accordance with the invention;

FIG. 29 is a top plan view of the reinforced organ encasement band shown in FIG. 28, in accordance with the invention;

FIG. 30 is a front plan view of another embodiment of a reinforced organ encasement band, in accordance with the invention;

FIG. 31 is a top plan view of the reinforced organ encasement band shown in FIG. 30, in accordance with the invention;

FIG. 32 is a perspective view of another embodiment of a reinforced biological construct, i.e. a reinforced organ encasement jacket, in accordance with the invention;

FIG. 33 is a perspective view of an organ encasement jacket illustrating one embodiment of a force distribution profile provided by a reinforced organ encasement jacket, in accordance with the invention; and

FIG. 34 is a perspective view of another embodiment of a reinforced biological construct, i.e. a reinforced organ encasement jacket having user modulated force distribution means, in accordance with the invention.

### DETAILED DESCRIPTION OF THE PREFERRED EMBODIMENT

Before describing the present invention in detail, it is to be understood that this invention is not limited to particularly exemplified apparatus, systems, materials, compositions, structures or methods as such may, of course, vary. Thus, although a number of apparatus, systems, materials, compositions, structures and methods similar or equivalent to those described herein can be used in the practice of the present invention, the preferred apparatus, systems, materials, compositions, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “an active” includes two or more such actives and the like.

Further, ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10”, as well as “greater than or equal to 10” is also disclosed.

### Definitions

The terms “construct” and “prosthesis” are used interchangeably herein, and mean and include a structure or system that is configured for placement on biological tissue on or in an organ and/or tissue related thereto. As discussed in detail herein, upon placement of a biological construct of the invention to a damaged mammalian organ and/or tissue related thereto, the biological construct induces “modulated healing” and, in some embodiments, “adaptive regeneration and/or remodeling”, as defined herein.

The term “biocompatible”, as used herein, means a device or material that is substantially non-immunogenic in an in vivo environment, and is not substantially rejected by a recipient's physiological system, i.e. non-antigenic.

The terms “extracellular matrix” and “ECM” are used interchangeably herein, and mean and include a collagen-rich substance that is found in between cells in mammalian tissue, and any material processed therefrom, e.g. decellularized ECM. According to the invention, the ECM material can be derived from various mammalian tissue sources including, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

The ECM material can thus comprise, without limitation, small intestine submucosa (SIS), urinary bladder submucosa (UBS), stomach submucosa (SS), central nervous system tissue, dermal extracellular matrix, subcutaneous extracellular matrix, gastrointestinal extracellular matrix, i.e. large and small intestines, tissue surrounding growing bone, placental extracellular matrix, omentum extracellular matrix, epithelium of mesodermal origin, i.e. mesothelial tissue, cardiac extracellular matrix, e.g., pericardium and/or myocardium, kidney extracellular matrix, pancreas extracellular matrix, lung extracellular matrix, and combinations thereof. The ECM can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM can also be derived from basement membrane of mammalian tissue/organs, including, without limitation, bladder, “urinary basement membrane (UBM)”, liver, i.e. “liver basement membrane (LBM)”, and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM can also be derived from other sources, including, without limitation, collagen from plant sources and synthesized extracellular matrices, i.e. cell cultures.

The term “ECM composition”, as used herein, means and includes a composition comprising at least one ECM.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The term “ECM-mimicking material”, as used herein, means and includes a biocompatible and biodegradable biomaterial that induces neovascularization and bioremodeling of tissue in vivo, i.e. when disposed proximate damaged biological tissue. The term “ECM-mimicking material” thus includes, without limitation, ECM-mimicking polymeric biomaterials; specifically, poly(glycerol sebacate) (PGS).

The term “ECM-mimicking material” also includes, without limitation, a “hydrogel” and/or “collagen” that is enhanced with an ECM component, such as TGF-β, or a ligand.

The term “biologically active agent”, as used herein, means and includes an agent that induces or modulates a physiological or biological process, or cellular activity, e.g., induces proliferation, and/or growth and/or regeneration of tissue.

The term “biologically active agent” thus means and includes, without limitation, the following growth factors: platelet derived growth factor (PDGF), epidermal growth factor (EGF), transforming growth factor alpha (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor alpha (TNF-α), and placental growth factor (PLGF).

The term “biologically active agent” also means and includes, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells. The group also comprises cardiomyocytes, myoblasts, monocytes, parenchymal cells, epithelial cells, endothelial cells, mesothelial cells, fibroblasts, osteoblasts, chondrocytes, exogenous cells, endogenous cells, macrophages, capillary endothelial cells, autologous cells, xenogenic cells, allogenic cells, and cells derived from any of the three germ layers including the endoderm, mesoderm and ectoderm.

The term “biologically active agent” also means and includes, without limitation, the following biologically active agents (referred to interchangeably herein as a “protein”, “peptide” and “polypeptide”): collagen (types I-V), proteoglycans, glycosaminoglycans (GAGs), glycoproteins, cytokines, cell-surface associated proteins, cell adhesion molecules (CAM), endothelial ligands, matrikines, cadherins, immuoglobins, fibril collagens, non-fibrillar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, fibulin II, integrins, transmembrane molecules, thrombospondins, ostepontins, and angiotensin converting enzymes (ACE).

The term “biologically active composition”, as used herein, means and includes a composition comprising at least one “biologically active agent.”

The term “pharmacological agent”, as used herein, means and includes an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs: laboratory animals, such as mice, rats and guinea pigs; fish; reptiles; zoo and wild animals; and the like.

The term “pharmacological agent” thus means and includes, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The term “pharmacological agent” thus includes, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti-VEGFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The term “pharmacological agent” further means and includes the following Class I-Class V anti-arrhythmic agents: (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem and (Class V) adenosine and digoxin.

The term “pharmacological agent” further means and includes, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

As indicated above, the term “pharmacological agent” further means and includes, without limitation, an anti-inflammatory.

The term “anti-inflammatory”, as used herein, means and includes an agent that prevents or treats biological tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenol one, methenol one acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “pharmacological composition”, as used herein, means and includes a composition comprising a “pharmacological agent”.

The term “therapeutically effective”, as used herein, means that the amount of an “biologically active agent”, “biologically active composition”, “pharmacological agent” and/or “pharmacological composition” administered to a mammalian organ or biological tissue is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “prevent” and “preventing” are used interchangeably herein, and mean and include reducing the frequency or severity of a disease or condition. The term does not require an absolute preclusion of the disease or condition. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition, or disorder. The terms include “active treatment”, i.e. treatment directed specifically toward the improvement of a disease, pathological condition, or disorder, and “causal treatment”, i.e. treatment directed toward removal of the cause of the associated disease, pathological condition, or disorder.

The terms “treat” and “treatment” further include “palliative treatment”, i.e. treatment designed for the relief of symptoms rather than the curing of the disease, pathological condition, or disorder, “preventative treatment”, i.e. treatment directed to minimizing or partially or completely inhibiting the development of the associated disease, pathological condition, or disorder, and “supportive treatment”, i.e. treatment employed to supplement another specific therapy directed toward the improvement of the associated disease, pathological condition, or disorder.

The terms “optional” and “optionally” mean that the subsequently described event, circumstance, or material may or may not occur or be present, and that the description includes instances where the event, circumstance, or material occurs or is present and instances where it does not occur or is not present.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As indicated above, the present invention is directed to biocompatible biological constructs that can be engineered into a variety of shapes and employed to treat and/or support damaged or diseased mammalian organs and/or tissue related thereto, and methods for employing same to treat and/or support damaged or diseased mammalian organs and/or tissue related thereto. In some embodiments of the invention, the biological constructs can thus be deemed constraining devices.

As discussed in detail below, in some embodiments of the invention, the biological constructs are configured to induce “modulated healing” of damaged mammalian organs and/or tissue associated therewith, including modulated inflammation of the damaged tissue and/or neovascularization, host tissue proliferation, bioremodeling, and regeneration of tissue and associated structures with site-specific structural and functional properties, when disposed proximate thereto.

In some embodiments of the invention, the biological constructs are configured to induce “adaptive regeneration” of a damaged mammalian organ; particularly, a mammalian heart, including induced hypertrophy and, thereby, modulation of organ function, when disposed proximate thereto.

In some embodiments, the biological constructs comprise a planar member, such as disclosed in Co-Pending application Ser. No. 14/554,730.

In some embodiments of the invention, the biological constructs comprise a jacket that is configured to encase a preselected region of a mammalian organ, i.e. an organ constrain device.

In some embodiments of the invention, the biological constructs comprise a band that is similarly configured to encase a preselected region of a mammalian organ.

In some embodiments of the invention, the biological constructs comprise a mesh fiber member.

In some embodiments of the invention, the biological constructs comprise a substantially solid member or sheet.

In some embodiments of the invention, the biological constructs comprise an ECM composition. In a preferred embodiment of the invention, the ECM composition includes at least one ECM material derived from a mammalian tissue source.

According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety. The mammalian tissue sources include, without limitation, the small intestine, large intestine, stomach, lung, liver, kidney, pancreas, amniotic membrane, placenta, heart, bladder, prostate, tissue surrounding growing enamel, tissue surrounding growing bone, and any fetal tissue from any mammalian organ.

The mammalian tissue can thus comprise, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, omentum extracellular matrix, cardiac extracellular matrix, e.g., pericardium and/or myocardium, kidney extracellular matrix, pancreas extracellular matrix, lung extracellular matrix, and combinations thereof. The ECM can also comprise collagen from mammalian sources.

In some embodiments, the mammalian tissue source comprises mesothelial tissue.

In a preferred embodiment, the mammalian tissue source comprises an adolescent mammalian tissue source, e.g. tissue derived from a porcine mammal less than 3 years of age.

The ECM can also be derived from the same or different mammalian tissue sources, as disclosed in Co-Pending application Ser. Nos. 13/033,053 and 13/033,102; which are incorporated by reference herein.

According to the invention, the ECM material can also comprise mixed solid particulates. The ECM material can also be formed into a particulate and fluidized, as described in U.S. Pat. Nos. 5,275,826, 6,579,538 and 6,933,326, to form a mixed emulsion, mixed gel or mixed paste.

According to the invention, the ECM can also be sterilized via applicant's proprietary novasterilis process disclosed in Co-Pending U.S. application Ser. No. 13/480,205; which is expressly incorporated by reference herein in its entirety.

In some embodiments of the invention, the biological constructs comprise an ECM-mimicking polymeric composition.

In some embodiments, the ECM-mimicking composition comprises poly(glycerol sebacate) (PGS).

As set forth in Co-Pending application Ser. No. 14/554,730, PGS exhibits numerous beneficial properties that provide several beneficial biochemical actions or activities; particularly, ECM-mimicking properties and actions.

Indeed, it has been found that PGS induces tissue remodeling and regeneration when administered proximate to damaged tissue, thus, mimicking the seminal regenerative properties of ECM and, hence, an ECM composition formed therefrom. The mechanism underlying this behavior is deemed to be based on the mechanical and biodegradation kinetics of the PGS. See Sant, et al., Effect of Biodegradation and de novo Matrix Synthesis on the Mechanical Properties of VIC-seeded PGS-PCL scaffolds, Acta. Biomater., vol. 9(4), pp. 5963-73 (2013).

In some embodiments of the invention, the ECM-mimicking composition comprises PGS and PCL. According to the invention, the addition of PCL to the ECM-mimicking composition enhances the structural integrity and modulates the degradation of the composition.

In some embodiments of the invention, the biological constructs comprise an ECM-PGS composition, e.g. 50% ECM/50% PGS.

In some embodiments, the ECM-PGS composition further comprises PCL.

In some embodiments of the invention, the biological constructs comprise a PVA composition comprising polyvinyl alcohol.

In some embodiments of the invention, the biological constructs comprise at least one additional biologically active agent, i.e. an agent that induces or modulates a physiological or biological process, or cellular activity, e.g., induces proliferation, and/or growth and/or regeneration of tissue, including, without limitation, the aforementioned biologically active agents.

In some embodiments, the biologically active agent thus comprises a cell selected from the group comprising, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells.

In some embodiments, the biologically active agent comprises a growth factor selected from the group comprising, without limitation, transforming growth factor alpha (TGF-α), transforming growth factor beta (TGF-β), fibroblast growth factor-2 (FGF-2), basic fibroblast growth factor (bFGF), vascular epithelial growth factor (VEGF), insulin-like growth factor (IGF) and hepatic growth factor (HGF).

In some embodiments, the biologically active agent comprises a protein selected from the group comprising, without limitation, collagen (types I-V), proteoglycans, glycosaminoglycans (GAGs), glycoproteins, cytokines, cell-surface associated proteins, and cell adhesion molecules (CAMs).

In some embodiments of the invention, the biologically active agent is selected from the group comprising RNAs, DNAs and nucleic acid sequence compounds that alter the genetic expression of host cells within the myocardium and epicardium, including, without limitation, progenitor cells, fibroblasts and mesothelial cells. The later may or may not use virally directed genetic coding that function in genome altering sequences or act upon epigenetic areas to activate or deactivate genes.

In some embodiments of the invention, the biological constructs comprise at least one pharmacological agent or composition, i.e. an agent, drug, compound, composition of matter or mixture thereof, including its formulation, that is capable of producing a desired biological effect in vivo, e.g., stimulation or suppression of apoptosis, stimulation or suppression of an immune response, including, without limitation, the aforementioned pharmacological agents.

In some embodiments, the pharmacological agent or composition is thus selected from the group comprising, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. In particular, Applicant has found that when a statin is added to ECM (wherein a statin augmented ECM composition is formed) and the statin augmented ECM composition is administered to damaged tissue, the statin interacts with the cells recruited by the ECM, wherein the statin augmented ECM composition modulates inflammation of the damaged tissue by modulating several significant inflammatory processes, including restricting expression of monocyte chemoattractant protein-1 (MCP-1) and chemokine (C—C) motif ligand 2 (CCR2).

The properties and beneficial actions are discussed in detail in Applicant's Co-Pending application Ser. No. 13/328,287, filed on Dec. 16, 2011, Ser. No. 13/373,569, filed on Sep. 24, 2012 and Ser. No. 13/782,024, filed on Mar. 1, 2013; which are incorporated by reference herein in their entirety.

Additional suitable pharmacological agents and compositions that can be delivered within the scope of the invention are disclosed in Pat. Pub. Nos. 20070014874, 20070014873, 20070014872, 20070014871, 20070014870, 20070014869, and 20070014868; which are expressly incorporated by reference herein in its entirety.

According to the invention, the biologically active and pharmacological agents referenced above can comprise various forms. In some embodiments of the invention, the biologically active and pharmacological agents, e.g. simvastatin, comprise microcapsules that provide delayed delivery of the agent contained therein.

In some embodiments, the biological constructs provide a single-stage agent delivery profile, i.e. comprise a single-stage delivery vehicle, wherein a modulated dosage of a biologically active and/or pharmacological agent is provided.

As set forth in Co-Pending application Ser. No. 14/554,730, the term “modulated dosage” and variants of this language generally refer to the modulation (e.g., alteration, delay, retardation, reduction, etc.) of a process involving different eluting or dispersal rates of an agent within biological tissue.

In some embodiments, the single-stage delivery vehicle comprises encapsulated particulates of a biologically active and/or pharmacological agent.

In some embodiments, the encapsulation composition comprises an ECM composition.

In some embodiments, the encapsulation composition comprises a biodegradable polymeric composition comprising a polymeric material selected from the group comprising, without limitation, polyglycolide (PGA), polylactide (PLA), polyepsilon-caprolactone, poly-dioxanone, poly lactide-co-glycolide polysaccharides (e.g. starch and cellulose), proteins (e.g., gelatin, casein, silk, wool, etc.), one of the aforementioned hydrogels, and combinations thereof.

In some embodiments of the invention, the encapsulation composition comprises an ECM-mimicking composition.

In some embodiments of the invention, the encapsulation composition comprises an ECM-PGS composition.

In some embodiments of the invention, the biological constructs provide a multi-stage agent delivery profile, i.e. comprise a multi-stage agent delivery vehicle, wherein a plurality of biologically active and/or pharmacological agents are administered via a modulated dosage.

In some embodiments of the invention, the biological constructs further comprise at least one coating.

In a preferred embodiment, the coatings of the invention comprise a biologically active composition.

In some embodiments of the invention, the biologically active compositions and, hence, coatings of the invention include one of the aforementioned biologically active or pharmacological agents.

In some embodiments of the invention, the coating is disposed proximate or on the biological construct encasement surface, as defined herein.

In some embodiments, the coating is disposed proximate or on the exterior surface of the biological construct.

In some embodiments, the biological constructs comprise multiple coatings having varying biologically active and/or pharmacological agents and/or properties, e.g. a first coating comprising a growth factor and a second coating comprising pharmacological agent.

In some embodiments, coatings comprising a biologically active and/or pharmacological agent comprise modulated degradation kinetics, wherein gradual degradation of the coating provides a controlled release of the biologically active and/or pharmacological agent.

In some embodiments, the biologically active composition comprises an ECM composition of the invention. In some embodiments, wherein the ECM composition comprises a biologically active and/or pharmacological agent, the ECM composition coating is configured to provide at least one biologically active and/or pharmacological agent delivery profile, as defined herein.

In some embodiments, the ECM coating is configured to provide a delivery gradient of various biologically active and/or pharmacological agent delivery profiles.

In some embodiments, the biologically active composition comprises an ECM-mimicking composition of the invention.

In some embodiments, the biologically active composition comprises an ECM-PGS composition of the invention.

In some embodiments, the biologically active composition comprises a polymeric composition comprising at least one biocompatible polymeric material.

According to the invention, the polymeric material can comprise, without limitation, polyglycolide (PGA), polylactide (PLA), polyepsilon-caprolactone (PCL), poly dioxanone (a polyether-ester), poly lactide-co-glycolide, polyamide esters, polyalkalene esters, polyvinyl esters, polyvinyl alcohol, and polyanhydrides.

The polymeric material can also comprise a hydrogel, including, without limitation, polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and β-hairpin peptide.

In some embodiments, the hydrogel is crosslinked via chemically and/or photocuring, e.g. ultraviolet light.

In some embodiments, the polymeric material is plasma treated to accommodate hygroscopic agents.

According to the invention, the coatings can additionally comprise a hydrogel, including, without limitation, polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and β-hairpin peptide. In some embodiments, the hydrogels are similarly configured to provide at least one biologically active and/or pharmacological agent delivery profile.

As indicated above, the biologically active compositions and, hence, coatings of the invention can also include one of the aforementioned biologically active or pharmacological agents.

A biologically active composition and, hence, coating of the invention can thus comprise an ECM composition coating comprising interleukin-10 (IL-10) and transforming growth factor beta (TGF-β) either alone, or in combination, to suppress the inflammatory reaction leading to a chronic immune response. During the chronic immune response IL-10 and TGF-β induce the expression of tissue inhibitor of metalloproteinase (TIMP), which inhibits matrix metalloproteinases (MMPs) that are responsible for ECM degradation during the inflammatory response. Additionally, IL-10 and TGF-β promote the recruitment of fibroblasts, which are the seminal cells responsible for ECM deposition and bioremodeling. As a result, IL-10, TGF-β, and the TIMPs concomitantly promote ECM deposition and preservation, which also augments “modulated healing.”

According to the invention, a biologically active composition and, hence, coating of the invention can also comprise an ECM composition comprising a pharmacological agent, such as an anti-inflammatory or antiviral, which provide a reinforcing anti-inflammatory effect either through direct reinforcement, i.e. targeting the same inflammatory signaling pathway, or indirect reinforcement, i.e. targeting an alternate inflammatory signaling pathway. An example of direct reinforcement includes, without limitation, a combination of IL-10, TGF-β and a glucocorticoid, all of which inhibit the expression of seminal inflammatory cytokine interleukin-1 (IL-1). An example of indirect reinforcement includes, without limitation, a combination of IL-10, TGF-β and an NSAID, (Non-steroidal anti-inflammatory drug) where IL-10 and TGF-β inhibit IL-1, and the NSAIDs inhibit the activity of both cyclooxygenase-1 (COX-1) and cyclooxygenase-2 (COX-2), and thereby, the synthesis of prostaglandins and thromboxanes.

According to the invention, in a preferred embodiment of the invention, upon deployment of a biological construct of the invention; particularly, a biological construct comprising ECM, an ECM-PGS composition and polymeric composition comprising an exogenously added biologically active agent, to a damaged mammalian organ and/or tissue associated therewith “modulated healing” is effectuated.

The term “modulated healing”, as used herein, and variants of this language generally refer to the modulation (e.g., alteration, delay, retardation, reduction, etc.) of a process involving different cascades or sequences of naturally occurring tissue repair in response to localized tissue damage or injury, substantially reducing their inflammatory effect. Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

For example, in some embodiments, the biological constructs are specifically formulated (or designed) to alter, delay, retard, reduce, and/or detain one or more of the phases associated with healing of damaged tissue, including, but not limited to, the inflammatory phase (e.g., platelet or fibrin deposition), and the proliferative phase when in contact with biological tissue.

In some embodiments of the invention, “modulated healing” means and includes the ability of a biological construct to restrict the expression of inflammatory components. By way of example, according to the invention, when a biological construct comprising a statin augmented ECM composition, i.e. a composition comprising an ECM and a statin, is disposed proximate damaged biological tissue, the biological construct restricts expression of monocyte chemoattractant protein-1 (MCP-1) and chemokine (C—C) motif ligand 2 (CCR2).

In some embodiments, “modulated healing” means and includes the ability of a biological construct to alter a substantial inflammatory phase (e.g., platelet or fibrin deposition) at the beginning of the tissue healing process. As used herein, the phrase “alter a substantial inflammatory phase” refers to the ability of a biological construct to substantially reduce the inflammatory response at an injury site when in contact with biological tissue.

In such an instance, a minor amount of inflammation may ensue in response to tissue injury, but this level of inflammation response, e.g., platelet and/or fibrin deposition, is substantially reduced when compared to inflammation that takes place in the absence of a biological construct of the invention.

The term “modulated healing” also refers to the ability of a biological construct to induce host tissue proliferation, bioremodeling, including neovascularization, e.g., vasculogenesis, angiogenesis, and intussusception, and regeneration of tissue structures with site-specific structural and functional properties.

Thus, in some embodiments, the term “modulated healing” means and includes the ability of a biological construct to modulate inflammation and/or induce host tissue proliferation and remodeling. Again, by way of example, according to the invention, when a biological construct comprising a statin augmented ECM composition is disposed proximate damaged biological tissue, the stain interacts with cells recruited by the ECM, wherein the biological construct modulates inflammation by, among other actions, restricting expression of monocyte chemoattractant protein-1 (MCP-1) and chemokine (C—C) motif ligand 2 (CCR2) and induces tissue proliferation, bioremodeling and regeneration of tissue structures with site-specific structural and functional properties.

By way of a further example, according to the invention, when a biological construct comprises a growth factor augmented ECM composition, i.e. a composition comprising an ECM and an exogenously added growth factor, e.g. TGF-β, and the construct is disposed proximate damaged biological tissue, the growth factor similarly interacts with the ECM and cells recruited by the ECM, wherein the biological construct modulates inflammation and induces tissue proliferation, bioremodeling and regeneration of tissue.

In some embodiments, when a mesh biological construct is in contact with biological tissue, modulated healing is effectuated through the structural features of a mesh biological construct. The structural features provide the spatial temporal and mechanical cues to modulate cell polarity and alignment. The structural features further modulate cell proliferation, migration and differentiation thus modulating the healing process.

In some embodiments, the mesh biological constructs comprise an anisotropic fiber structure providing spatial temporal and mechanical cues.

Accordingly, the mesh biological constructs of the invention provide an excellent means for treating damaged or diseased organs and tissue, including closing and maintaining closure of openings in biological tissue, e.g., closure of openings in tissue after surgical intervention.

Referring now to FIG. 1, there is shown one embodiment of a biocompatible strand 12a that can be employed to form biological constructs of the invention. As indicated above, the strand 12a can comprise various dimensions, e.g., length, circumference, etc., to accommodate various fiber construct and mesh fiber member structures and applications.

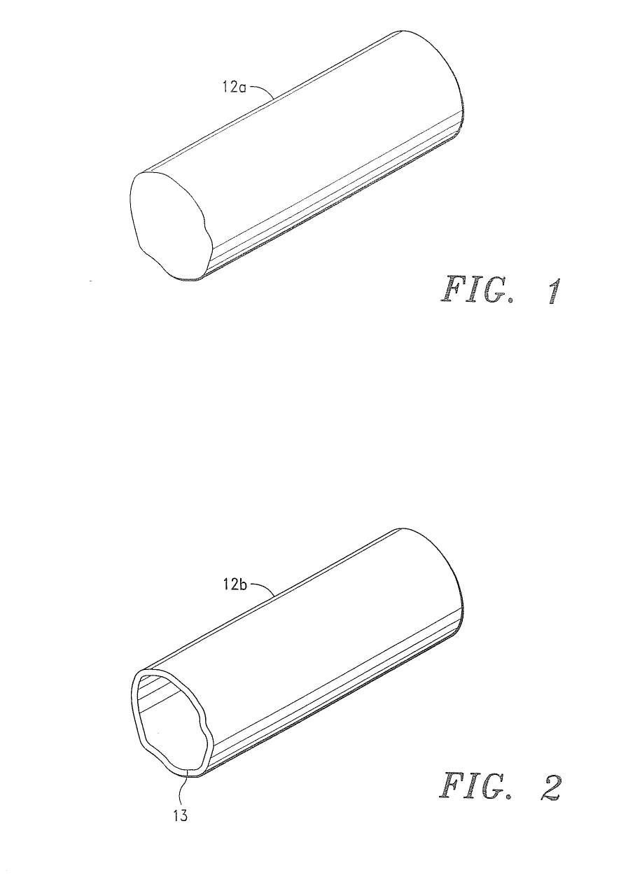

Referring now to FIG. 2, there is shown another embodiment of a biocompatible strand 12b that can be employed to form biological constructs of the invention. As illustrated in FIG. 2, the strand 12b includes a luminal cavity 13.

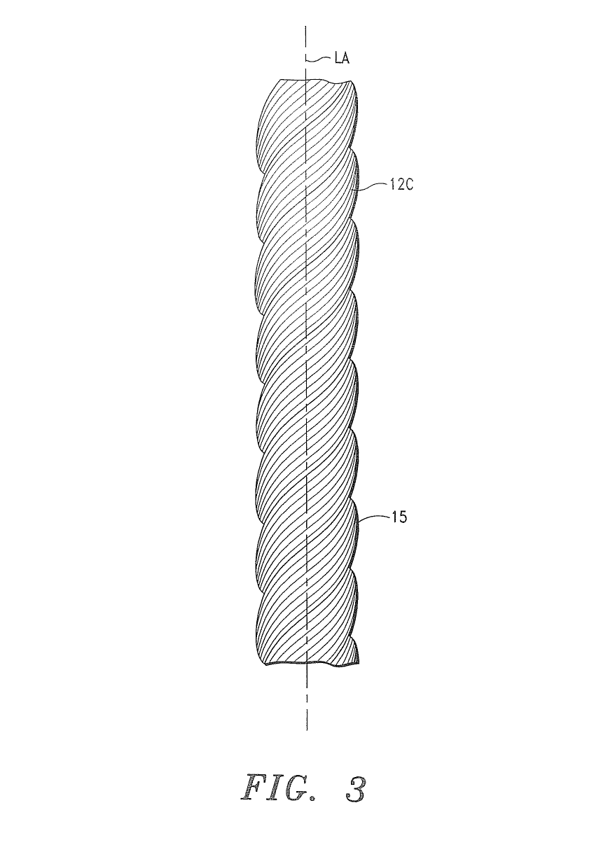

Referring now to FIG. 3, there is shown one embodiment of a fiber construct 15 that can be employed to form biological constructs of the invention. As illustrated in FIG. 3, the fiber construct 15 comprises a plurality of strands 12c, arranged in a substantially braided structure.

According to the invention, the fiber construct 15 can similarly comprise various dimensions to accommodate various mesh fiber member structures and application.

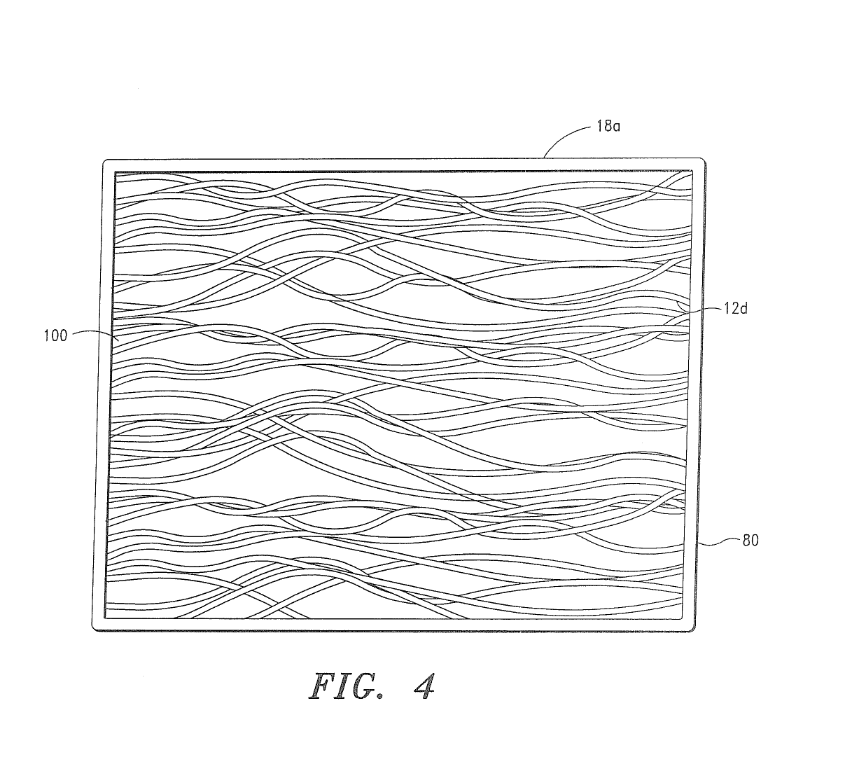

Referring now to FIG. 4, there is shown one embodiment of the mesh biological construct 18a of the invention. As illustrated in FIG. 4, the mesh biological construct 18a comprises a plurality of interwoven or intersecting strands 12d. As further illustrated in FIG. 4, the mesh biological construct 18a further comprises a constraining edge or border 80 that forms an internal fiber region 100.

According to the invention, the mesh biological construct 18a can also comprise a plurality of fiber constructs, such as construct 15 as shown in FIG. 3.

Referring now to FIG. 5, there is shown another embodiment of a mesh biological construct 18b. As illustrated in FIG. 5, in this embodiment, the mesh biological construct 18b comprises a plurality of intertwined strands 12e.

In the illustrated embodiment, each strand 12e, is oriented at an angle (“α”) in the range of approximately 0-89° relative to a line corresponding to the plane defined by the linear axis (“LA”) of the mesh construct 18b.

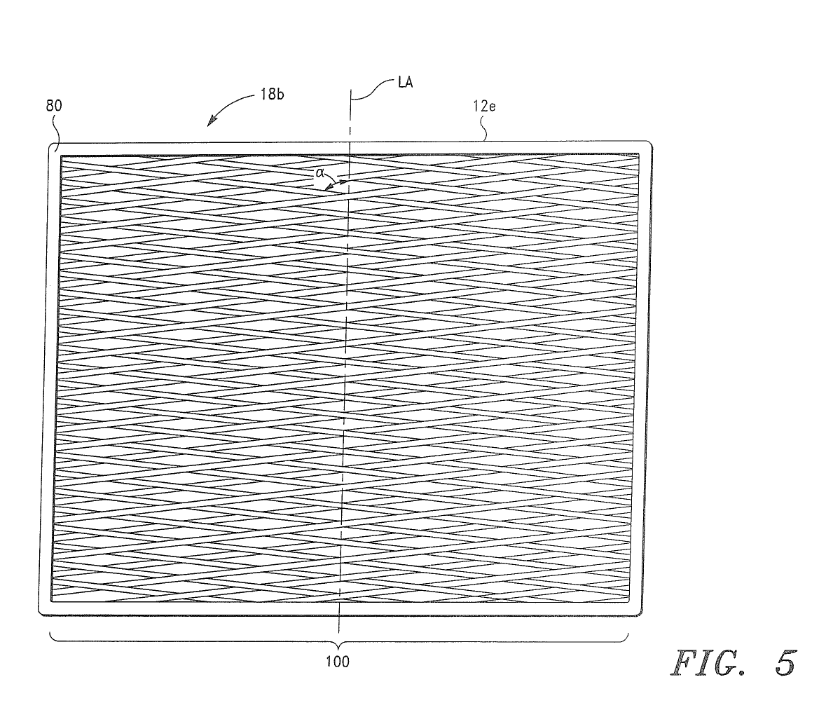

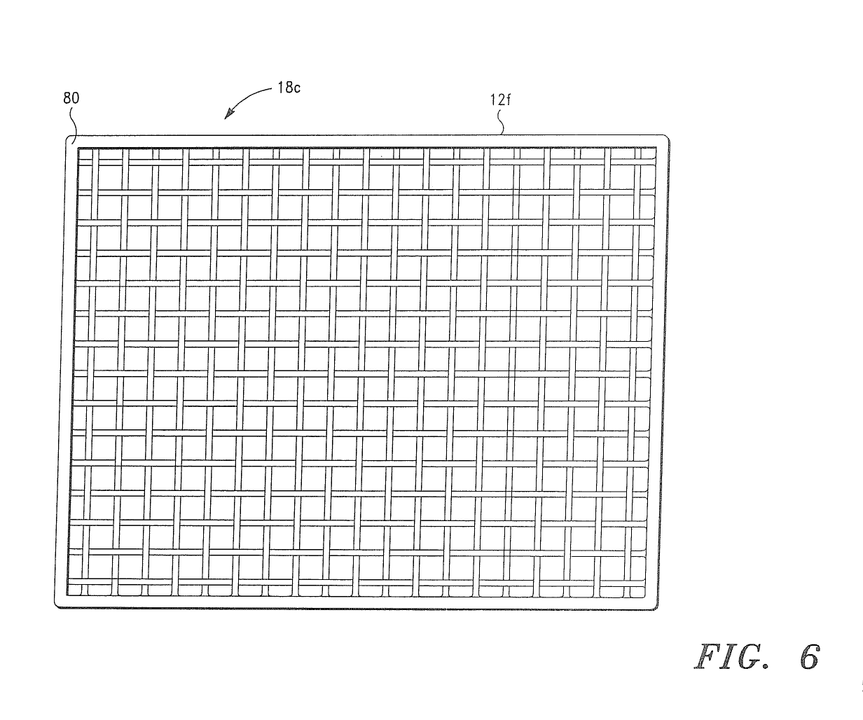

Referring now to FIG. 6, there is shown another embodiment of a mesh biological construct 18c having a plurality of substantially perpendicular interwoven or intersecting strands 12f.

Referring now to FIG. 7, there is shown another embodiment of the mesh biological construct 18e having a plurality of intertwined, randomly oriented strands 12g.

It is understood that the mesh biological construct patterns shown in FIGS. 4-7 are merely examples of the various mesh patterns that can be employed within the scope of the invention. The mesh patterns shown in FIGS. 4-7 should thus not be construed as limiting the scope of the invention in any manner.

As will be readily appreciated by one having ordinary skill in the art, the mesh biological constructs of the invention can be readily employed in various medical procedures, including, without limitation, treatment of coronary and peripheral vascular disease (PVD) in cardiovascular vessels, including, but not limited to, iliacs, superficial femoral artery, renal artery, tibial artery, popliteal artery, etc., deep vein thromboses (DVT), vascular bypasses, and coronary vascular repair.

The mesh biological constructs can also be readily employed to construct a pouch that is configured to encase an ECM or pharmacological composition, or medical instrument or device, such as a pacemaker, therein. Illustrative pouch configurations are disclosed U.S. Pat. No. 8,758,448 and Applicant's Co-pending U.S. application Ser. Nos. 13/573,566 and 13/896,424, which are incorporated by reference herein in their entirety.

In some embodiments, the mesh biological constructs are configured to form an organ encasement jacket (or sock) or band that is designed and configured to encase a preselected region of a mammalian organ.

In a preferred embodiment of the invention, the mammalian organ comprises a heart.

According to the invention, the mammalian organ can also comprise an organ selected from the group comprising, the small intestine, large intestine, stomach, lung, liver, kidney, pancreas, placenta, heart, bladder and prostate.

Thus, although the organ encasement jackets and bands of the invention are described herein in connection with encasing a preselected region of a mammalian heart, the organ jackets and bands can also readily be employed to encase a preselected region of any of the aforementioned organs.

In a preferred embodiment of the invention, the organ encasement jackets and/or bands of the invention are adjustable on the mammalian heart to conform to an external topography of the heart and, preferably, assume a maximum adjusted volume. In a preferred embodiment, the jackets and/or bands are also configured to constrain circumferential expansion of the heart beyond the maximum adjusted volume during diastole and to permit unimpeded contraction of the heart during systole.

As indicated above, in some embodiments, the organ encasement jackets and/or bands comprise mesh biological constructs.

According to the invention, the organ encasement jackets and bands of the invention can also comprise non-mesh biological constructs, i.e. formed from conventional sheet materials, such as disclosed in U.S. Pat. No. 8,758,448 and Co-Pending application Ser. No. 13/896,424; which are incorporated by reference herein.

Referring now to FIG. 12, there is shown a depiction of a normal human heart 200. As illustrated in FIG. 12, the heart 200 comprises four internal chambers; the right atrium 224, right ventricle 220, left atrium 210 and left ventricle 204. The left atrium 210 and left ventricle 204 are divided laterally by the circumflex artery 208, while the right atrium 224 and the right ventricle 220 are divided laterally by the right coronary artery 222.

As further illustrated in FIG. 12, the heart 200 comprises the right pulmonary veins 226 and the left pulmonary veins 212, which direct blood into the left atrium 210. The heart 200 further comprises the inferior vena cava 216 which directs deoxygenated blood to the right atrium 224. The heart 200 also comprises the small cardiac vein 218 and the left coronary artery 206 disposed on the surface of myocardium 230.

As further illustrated in FIG. 12, for purposes of this disclosure, the heart 200 is divided into two sub-regions, comprising a lower region L′ and an upper region U′, which are divided circumferentially by the right coronary artery 222 and circumflex artery 208. The lower region L′ comprises the right ventricle 220, left ventricle 204, left coronary artery 206 and the small cardiac vein 218. The upper region U′ comprises the right atrium 224, left atrium 210, left pulmonary artery 214 and left pulmonary veins 212.

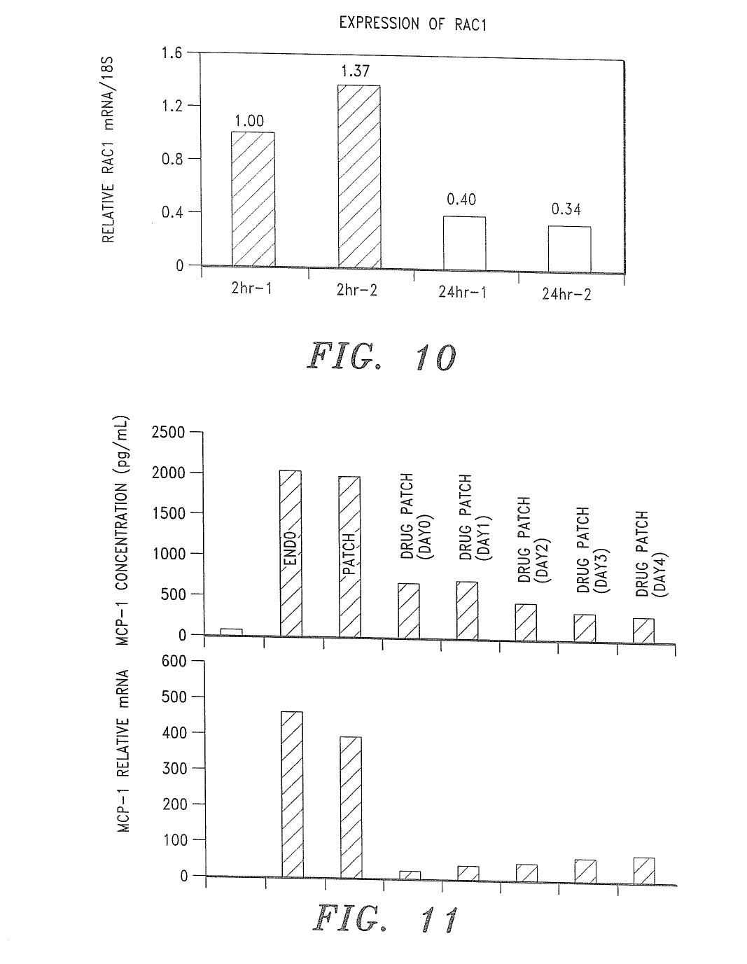

Referring now to FIG. 13, there is shown one embodiment of a biological construct in the form of an organ encasement jacket. As illustrated in FIG. 13, the organ encasement jacket 20 comprises an encasement surface 22 and an exterior surface 28.

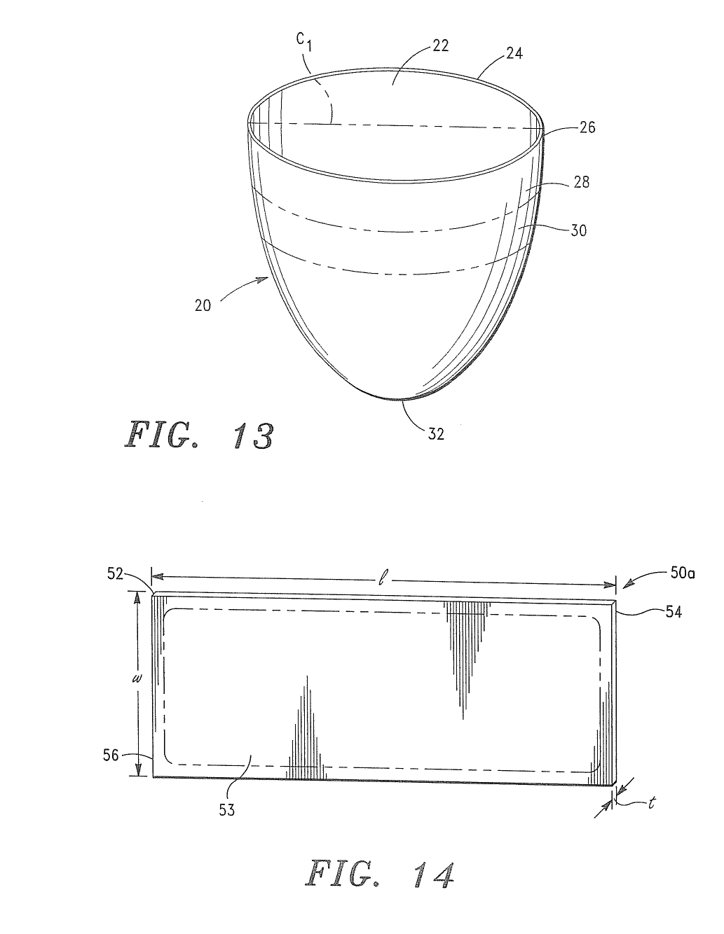

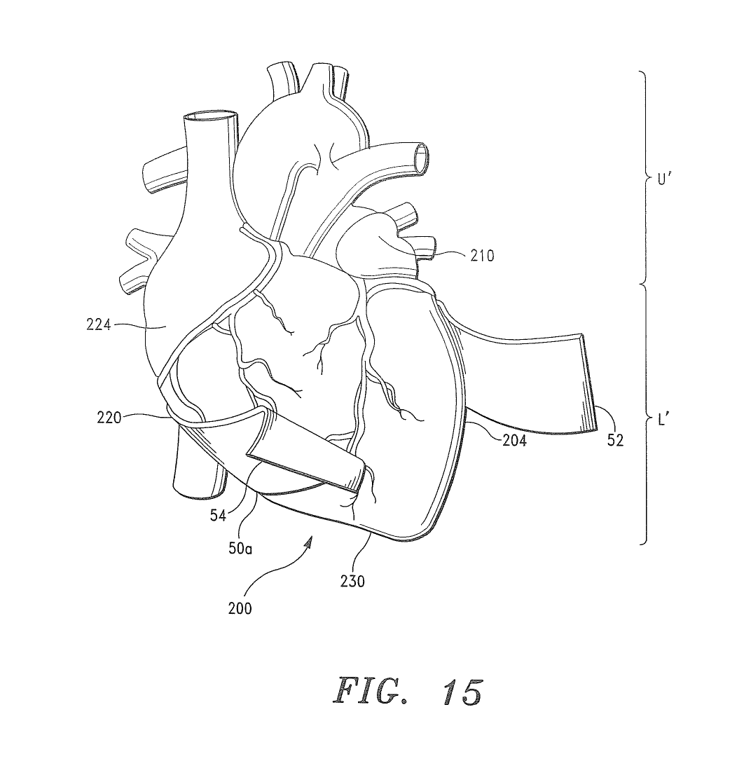

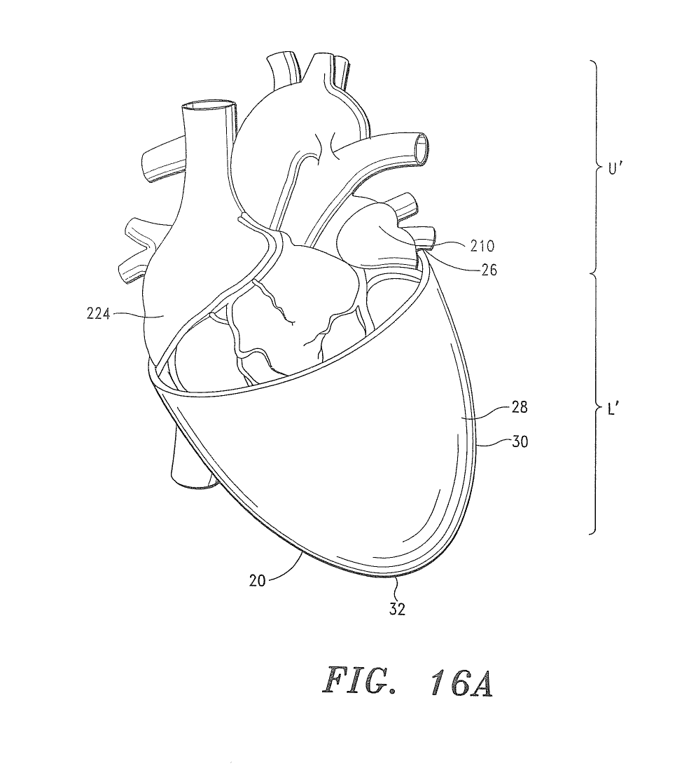

By the term “encasement surface,” as used in connection with an organ encasement jacket or band of the invention, it is meant to mean the surface of a biological construct that is disposed proximate the organ, e.g., heart 200, when encased by a biological construct of the invention. (See FIGS. 16A and 16B)

Referring back to FIG. 13, the organ encasement jacket 20 also comprises a proximal end 26 having an open configuration 24 comprising circumference C₁, a lateral mid region 30 and a distal end 32.

According to the invention, the distal end 32 can comprise a closed or open configuration. In a preferred embodiment, the distal end 32 comprises a closed configuration.

Referring now to FIG. 16A, there is shown one embodiment of organ displacement jacket 20 positioned over and, hence, disposed proximate lower region (L′) of heart 200. In a preferred embodiment, the jacket 20 is disposed over at least 80% of lower region L′.

Referring now to FIG. 14, there is shown one embodiment of a biological construct in the form of an organ encasement band. According to the invention, the organ encasement band 50a can comprise various lengths, widths and thicknesses to accommodate placement around a desired organ. The organ encasement band 50a can also comprise various edge configurations, e.g. curvilinear or straight ends.

In the illustrated embodiment, organ encasement band 50a further comprises a first end 52, second end 54, an encasement surface 53, length “l”, width “w”, thickness “t” and, optionally, an interior border 56 on the encasement surface 53 that is configured to facilitate sealing when the band 50 is disposed around and, hence proximate an organ region, such as illustrated in FIG. 16B.

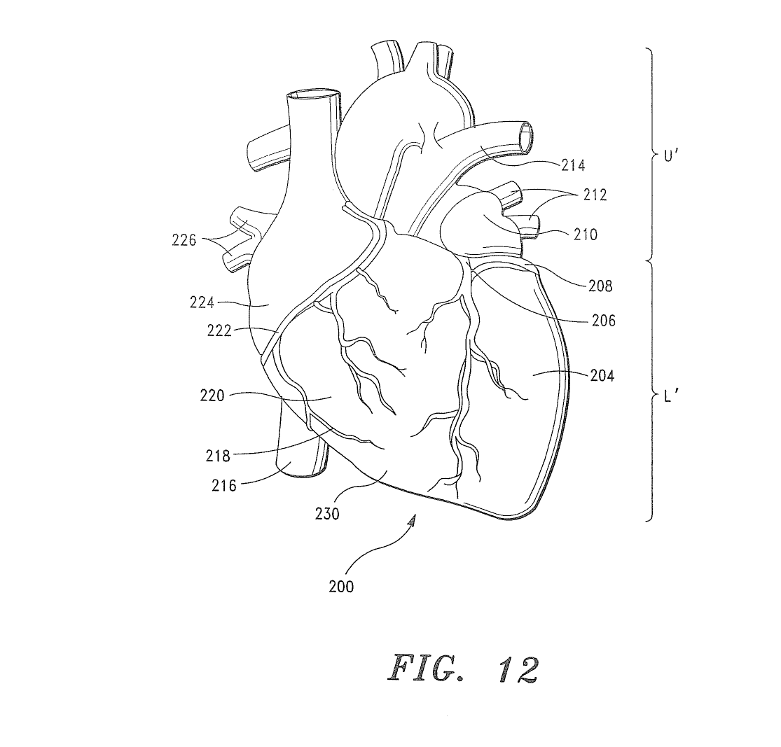

Referring now to FIG. 15, there is shown organ encasement band 50a disposed proximate a region of heart 200 prior to wrapping and, hence, encasing the heart 200. According to the invention, the band 50a is configured to fully wrap around a region of the heart 200, as illustrated in FIG. 16B.

In some embodiments, the band 50a has sufficient length “l” to wrap at least twice around a pre-selected region of heart 200.

Referring now to FIG. 16B, there is shown an embodiment of organ encasement band 50a wrapped around and, hence, encasing a pre-selected mid-region of heart 200.

According to the invention, after the organ encasement band 50a is wrapped around the heart 200 the ends 52, 54 can be attached to each other by various convention means, such as sutures and adhesive compositions. In a preferred embodiment, the ends 52, 54 of the band 50a are attached via sutures.

According to the invention, multiple organ encasement bands of the invention, including band 50a, can be wrapped around and, hence, positioned on the heart 200. Thus, in some embodiments, multiple organ encasement bands, e.g., two bands, are positioned on the same pre-selected region of heart 200.

In some embodiments, multiple organ encasement bands are positioned on different regions of the heart 200. In some embodiments, the bands intersect when positioned around the heart 200.

According to the invention, the organ encasement bands of the invention can be employed to wrap around and, hence, encase a region of the heart 200 in any configuration that does not disrupt the function of any major veins or arteries, such as the inferior vena cava 216, right pulmonary veins 226 and the left pulmonary veins 212.

Preferably, the organ encasement jackets and/or bands are sized and configured to at least partially seal a preselected region of the heart 200. In some embodiments, the jackets and/or bands are configured to hermetically seal a region of the heart 200.

According to the invention, the organ encasement jackets and/or bands of the invention can be secured proximate a mammalian organ, such as heart 200, by various conventional means, including sutures and/or an adhesive composition.

In some embodiments, the organ encasement jackets and/or bands are secured to a selective organ by sutures.

In some embodiments, the sutures comprise conventional sutures. In some embodiments, the sutures comprise one of the aforementioned polymeric compositions. In some embodiments, the sutures comprise one of the aforementioned ECM compositions.

In some embodiments, the organ encasement jackets and/or bands are secured to a selective organ by an adhesive composition.

In some embodiments, the adhesive composition comprises one of the aforementioned hydrogel compositions and/or fibrin gel compositions. In some embodiments, the adhesive composition comprises one of the aforementioned ECM-mimicking compositions.

In some embodiments, the adhesive composition comprises at least one of the aforementioned bioactive and/or pharmacological agents.

In some embodiments, the adhesive composition comprises one of the aforementioned photoinitiators to form a photoinitiator-augmented adhesive composition.

According to the invention, the photoinitiator-augmented adhesive composition can be configured to crosslink or polymerize when exposed to a sufficient wavelength of radiation.

According to the invention, suitable radiation wavelengths for crosslinking and/or curing the photoinitiator-augmented adhesive composition can comprise, without limitation, visible light; particularly, radiation in the range of approximately 380-750 nm, and ultraviolet (UV) light, particularly, radiation in the range of 10-400 nm, which includes extreme UV (10-121 nm), vacuum UV (10-200 nm), hydrogen lyman α-UV (121-122 nm), Far UV (122-200 nm), Middle UV (200-300 nm), Near UV (300-400 nm), UV-C (100-280 nm), UV-B (280-315 nm) and UV-A (315-400 nm) species of UV light.

As indicated above, the organ encasement jackets and/or bands of the invention can further comprise a coating that is disposed on the encasement surface and/or exterior surface.

Referring now to FIG. 17, there is shown an embodiment of an organ encasement jacket 20c′ positioned on heart 200, wherein the jacket 20c′ comprises a coating 34 disposed on the encasement surface 22.

According to the invention, the coatings of the invention, including coating 34, can be disposed on a portion of the encasement and/or exterior surface or the entire encasement and/or exterior surface. In a preferred embodiment, the coatings of the invention are disposed over at least 90% of the encasement or exterior surface.

According to the invention, the organ encasement jackets and/or bands of the invention can further comprise multiple coatings disposed on the encasement and/or exterior surface.

Referring now to FIG. 18, there is shown an embodiment of an organ encasement jacket 20c″ positioned on heart 200, wherein the jacket 20c″ comprises a first coating 34 disposed on the encasement surface 22 and a second coating 36 disposed on the exterior surface 28.

Referring now to FIG. 19, there is shown an embodiment of a coated organ encasement band of the invention. As illustrated in FIG. 19, the coated organ encasement band 38 comprises a base band member 40 having a coating 42 disposed on the encasement surface 22.

In a preferred embodiment, coatings 34, 36 and 42 comprise one of the aforementioned biologically active compositions.

According to the invention, when a biologically active composition of the invention is disposed on an organ encasement surface of a jacket or band of the invention, and the jacket or band is positioned proximate or on an organ, the biologically active composition similarly induces and, thereby enhances “modulated healing”, as defined herein, while the jacket and/or band similarly provides simultaneous structural support for the organ.

According to the invention, when the noted organ encasement jacket and/or band is positioned on an organ, the jacket and/or band also provides a physical stimuli, i.e. a compressive force, to the failing heart during systole to induce and modulate adaptive regeneration of the heart.

By the terms “adaptive regeneration” and “site specific adaptive regeneration (SSPAR)” it is meant to mean the process of inducing modulated healing of damaged organ tissue concomitantly with stress-induced hypertrophy of the organ, wherein the organ adaptively remodels. According to the invention, the stress-induced hypertrophy can result from any external stimuli, including, without limitation, physical, electrical and chemical stimuli.

As is well known in the art, hypertrophy is an adaptive response during post-infarction remodeling that offsets increased load, attenuates progressive dilatation, and stabilizes contractile function. See Martin, et al., Left Ventricular Remodeling After Myocardial Infarction, Circulation, vol. 101, pp. 2981-2988 (2000) and Pfeffer, et al., Ventricular Remodeling after Myocardial Infarction: Experimental Observations and Clinical Implications. Circulation. vol. 81, pp. 1161-1172 (1990).

Cardiac hypertrophy, in particular, is stimulated by a variety of biochemical and physical stimuli, wherein stress-induced hypertrophy in cardiomyocytes mimics hemodynamic load-induced hypertrophy.

Cardiac hypertrophy is transduced through a common mechanism involving the activation of protein kinase cascades. The receptors for norepinephrine (NE), endothelin-1 (ET-1), and angiopoietin II (Ang II) are similar and are coupled to Gq proteins. The activation of the Gq alpha subunit (Gqα) stimulates phospholipase Cβ, which in turn leads to the production of 1, 2 diacylglycerol and the activation of protein kinase C (PKC). Growth factors, including fibroblast growth factor (FGF), epidermal growth factor (EGF), platelet-derived growth factor (PDGF), insulin, and insulin-like growth factor (IGF-1), activate receptor tyrosine kinase, p21 ras, and mitogen-activated protein (MAP) kinase (extracellular regulated kinase or Jun N-terminal kinase). The activation of MAP kinase is a prerequisite for the transcriptional and morphological changes of myocyte hypertrophy.

In some embodiments, the terms “adaptive regeneration” and “site specific adaptive regeneration (SSPAR)” thus mean and include the ability of the organ encasement jacket and/or band of the invention to modulate one or more stages of acute and/or chronic inflammation, and their interplay with each other.

In some embodiments, the terms “adaptive regeneration” and “site specific adaptive regeneration (SSPAR)” mean and include the ability of the organ encasement jackets and/or bands of the invention to induce hypertrophy and, hence, modulate organ function.

By way of example, when an organ encasement jacket comprising an ECM composition and an ECM-mimicking composition coating (on the encasement surface) comprising a PGS-cerivastatin composition is disposed on a mammalian heart with an infarct caused systolic heart failure, the jacket induces modulated healing of the infarct while exerting a compressive force on and, hence, structural support to the failing heart. The compressive force exerted on the heart by the organ encasement jacket induces hypertrophy, stress-induced hypertrophy and normalizes the abnormal increase in heart size, while increasing intra-ventricular pressure to assist the heart during systole. The strength of ventricular contraction is also assisted by the organ encasement jacket, whereby ventricular contraction generates sufficient stroke volume and, thereby, normalized cardiac output.

According to the invention, the organ encasement jackets and/or bands of the invention can also comprise a biologically active composition coating on the exterior surface, which, when the organ encasement jackets and/or bands are positioned on an organ, will induce modulated healing and, in some instances, adaptive regeneration and/or remodeling of the organ and/or soft tissue surrounding the organ.

As is well known in the art, after heart surgery the surgeon will often elect to forgo stitching the pericardial incision to reduce the risk of cardiac tamponad. However, one of the seminal functions of the pericardium is lost; particularly, securing of the heart to the thoracic cavity via soft tissue.

According to the invention, by employing an organ encasement jacket having a biologically active composition coating of the invention on the exterior surface (separately or in conjunction with a coating on the encasement surface) after such surgery, the coated organ encasement jacket will induce modulated healing and, optimally, adaptive regeneration and/or remodeling of the heart and/or soft tissue surrounding the heart, which, among other advantages, aids in securing of the heart to the thoracic cavity.

According to the invention, a biologically active composition of the invention can also be applied directly to a surface of the organ (separately or in conjunction with a coating on the exterior surface of a jacket or band) prior to positioning of the jacket and/or band on the organ.

In some embodiments, the organ encasement jackets and/or bands of the invention comprise reinforcement means.

In a preferred embodiment, the reinforcement means comprises a biocompatible support structure.

According to the invention, the support structures of the invention can comprise various configurations.

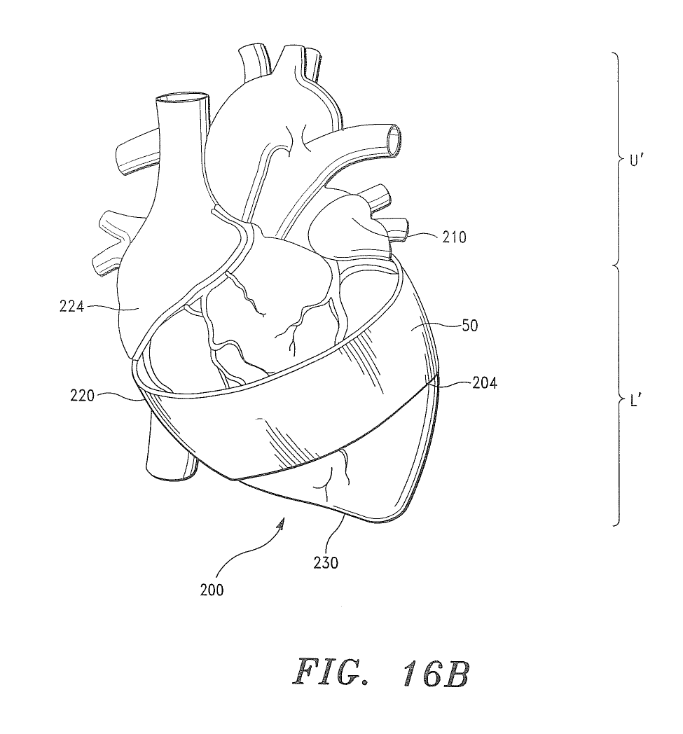

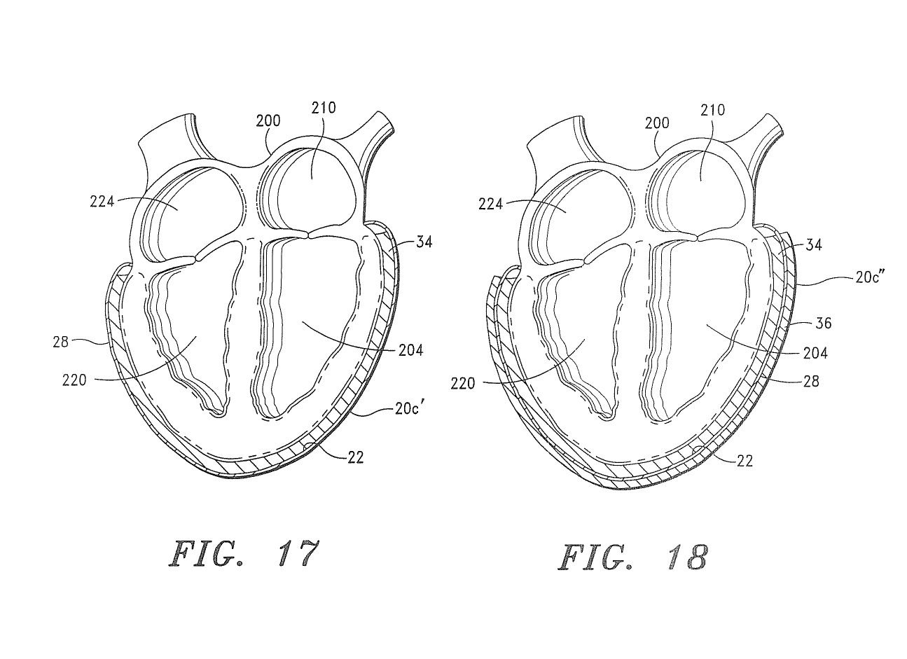

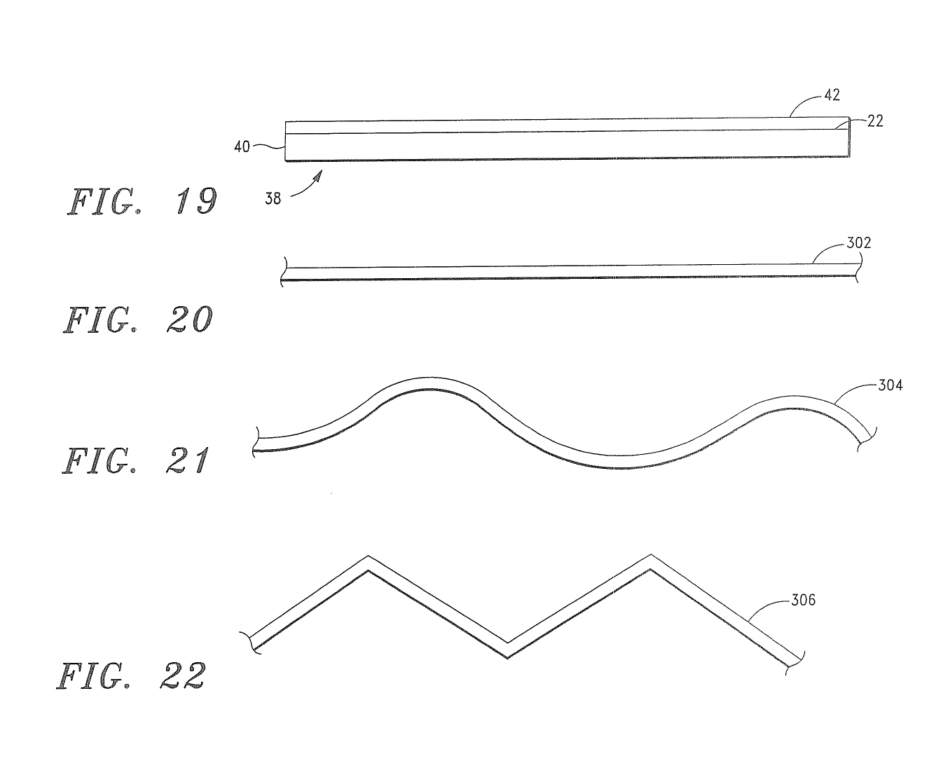

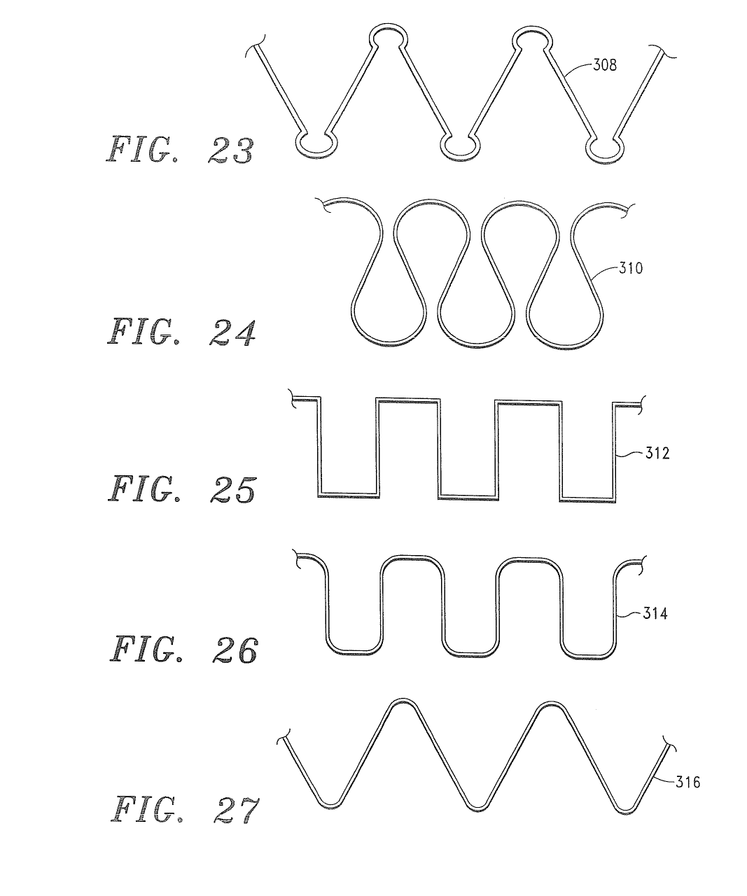

By way of example, referring now to FIGS. 20-28, a support structure of the invention can thus comprise, without limitation, a substantially linear structure, such as support structure 302 shown in FIG. 20, a curvilinear (or wave) support structure 304, such as shown in FIG. 21, a jagged or saw-tooth support structure 306 such as shown in FIG. 22, a curvilinear-tipped pyramid pattern support structure 308 such as shown in FIG. 23, a teardrop support structure 310 such as shown in FIG. 24, a rectangular support structure 312 such as shown in FIG. 25, a curvilinear-rectangular support structure 314 such as shown in FIG. 26 and a curvilinear-saw-tooth support structure 316 such as shown in FIG. 27.

In some embodiments, the support structure is incorporated in an organ encasement jacket and/or band of the invention.

In some embodiments, the support structure is disposed on a surface of the organ encasement jacket and/or band, e.g., an encasement surface 22 and/or exterior surface 28.

According to the invention, the support structure can be incorporated between plurality of sheet and/or mesh layers comprising the organ encasement jacket and/or band.

According to the invention, the organ encasement jackets and/or bands of the invention can include multiple support structures having the same or different configurations.

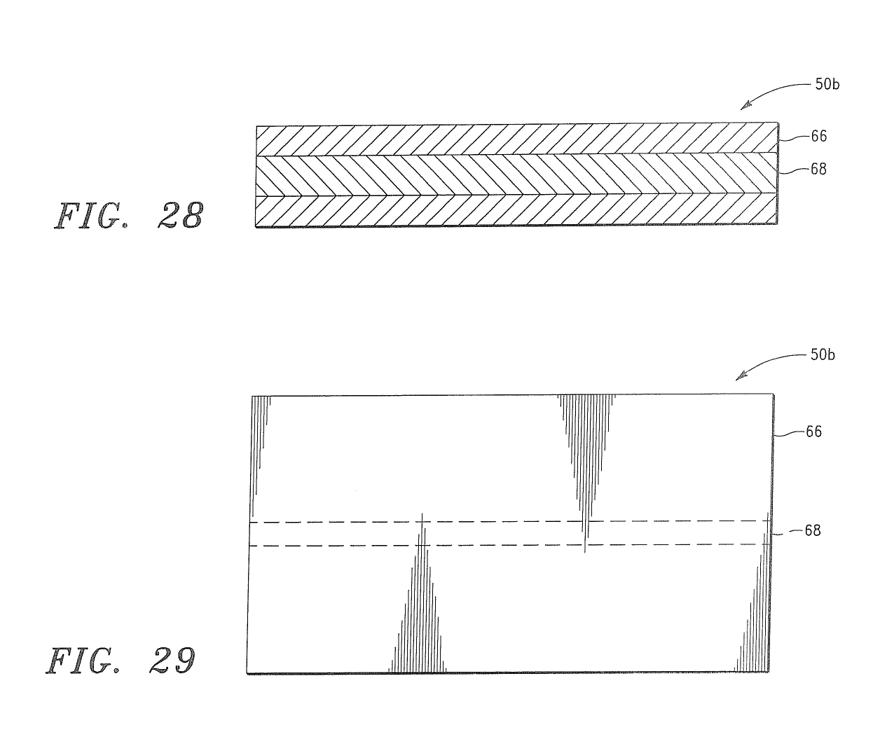

Referring now to FIG. 28, there is shown one embodiment of biological construct in the form of a reinforced organ encasement band. As illustrated in FIG. 28, the reinforced organ encasement band 50b includes a base band member 66 having a linear support member 68 disposed therein.

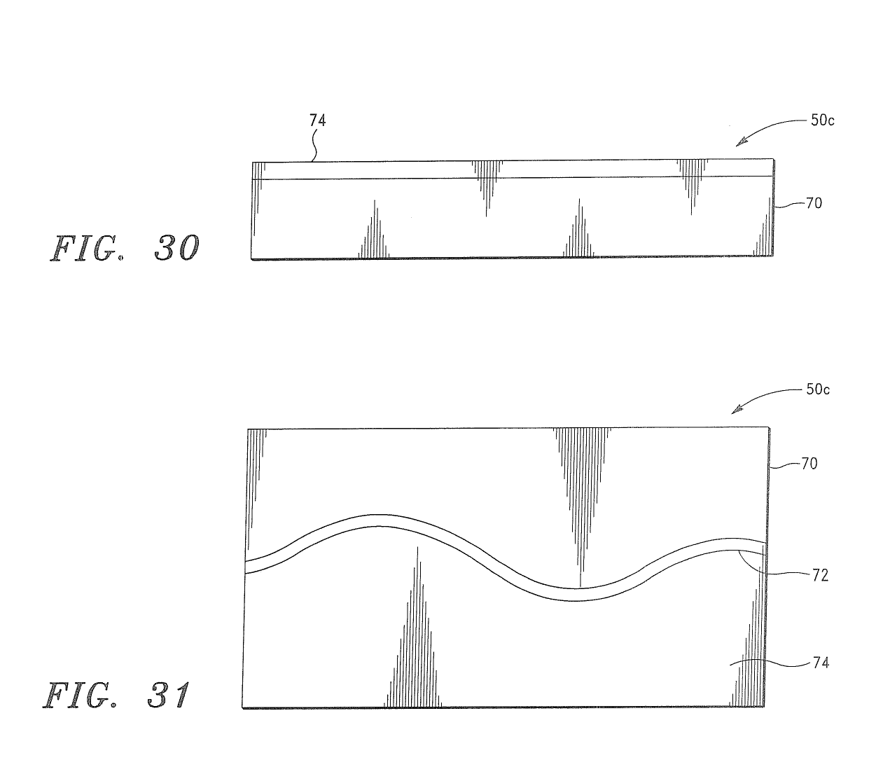

Referring now to FIG. 30, there is shown a further embodiment of a reinforced organ encasement band 50c having a base member 70 with a curvilinear support member 72 disposed on the outer surface 74.

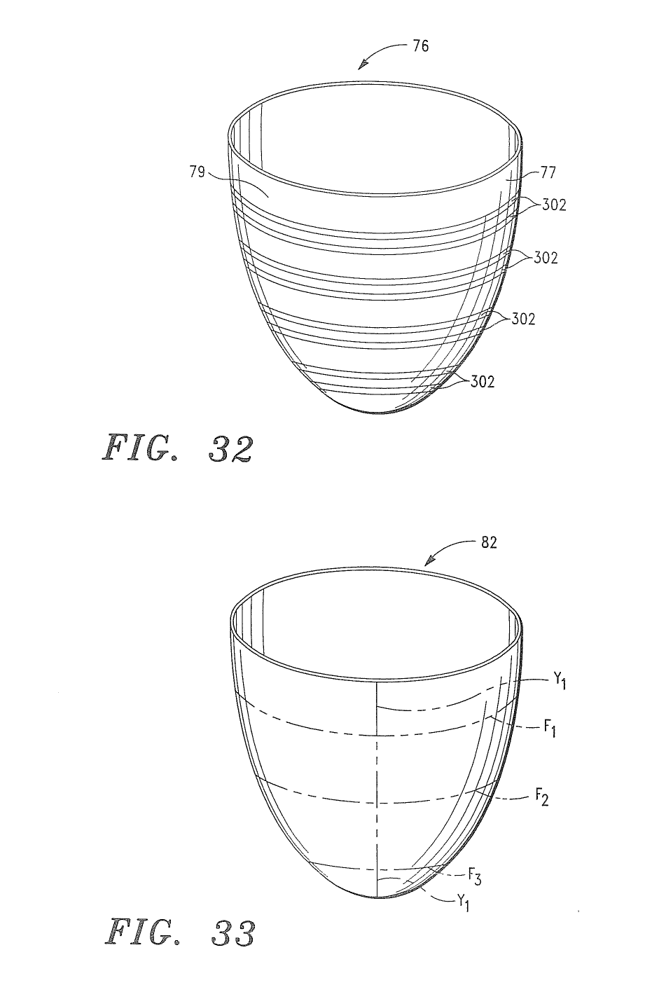

Referring now to FIG. 32, there is shown one embodiment of biological construct in the form of a reinforced encasement jacket. As illustrated in FIG. 32, the reinforced organ encasement jacket 76 comprises a base jacket member 77 having a plurality of linear support members 302 disposed on selective regions of the base member outer surface 79.

According to the invention, the support members 302, as well as the support members shown in FIGS. 21 through 27, can be positioned on the outer surface of jacket 76 at various pre-selected locations to provide a desired force pattern or profile.

According to the invention, the support members of the invention can be attached to a surface of an organ encasement jacket and/or band by various conventional means, including weaving the support structure into the jackets and/or bands, and the aforementioned sutures and/or adhesive compositions. The attachment means is of course dependent upon the configuration of the support structure.

According to the invention, the biocompatible support structures of the invention can comprise a biocompatible polymer selected from the group including, without limitation, Dacron®, Orlon®, Fortisan®, nylon, knitted polypropylene (e.g., Marlex®), microporous expanded-polytetrafluoroethylene (e.g., Gore-Tex®), Dacron reinforced silicone rubber (e.g., Silastlc®), polyglactin 910 (e.g., Vicryl®), polyester (e.g., Mersilene®) and polyglycolic acid (e.g., Dexon®).

The biocompatible support structures of the invention can also comprise a biocompatible metal selected from the group including, without limitation, stainless steel, titanium, cobalt-chromium-molybdenum alloy, cobalt-chrome-nickel alloy, and combinations and/or alloys thereof.

The biocompatible support structures of the invention can further comprise a biocompatible shape memory alloy, including, without limitation, nickel-titanium alloy (Nitinol®). In these embodiments, the biocompatible support structure is initially formed in a pre-deployment configuration or shape and subsequently heat-treated at a first temperature (i.e. shape set heat treatment) prior to attachment to an exterior surface of an organ encasement jacket and/or band of the invention.

After the reinforced jacket and/or band is placed at a desired position on an organ, such as a heart, the biocompatible support structure transitions to an austenitic phase (i.e. the temperature of the biocompatible support structure reaches and exceeds the Nitinol® transition temperature by virtue of the body temperature) and recovers its original pre-deployment shape, whereby the biocompatible support structure temporarily or permanently positions the reinforced jacket and/or band proximate host tissue of the organ with a pre-determined compressive force, “C_{f}”.

In some embodiments of the invention, the organ encasement jackets and/or bands are further configured to provide differential compressive forces, i.e. a differential force profile, when the jacket or band is positioned on or around an organ.

According to the invention, the organ encasement jackets of the invention can thus be configured to provide a circumferential force gradient along the vertical axis of the jacket.

Referring now to FIG. 33, there is shown an illustration of one embodiment of differential forces F₁, F₂, F₃ along the vertical axis Y₁ of a reinforced organ encasement jacket, in this instance, jacket 82 that can be provided by the reinforced organ encasement jackets of the invention.

According to the invention, multiple different forces at different regions on an organ encasement jacket or band of the invention can be provided by the organ encasement jackets and/or bands of the invention. By way of example, in some embodiments of the invention, an organ encasement jacket of the invention is configured to apply forces F₁ through Fₓ at multiple locations on the jacket.

According to the invention, the differential forces that are provided by the organ encasement jackets and/or bands of the invention can be provided by various means, including various solid and expandable support members.

In some embodiments, the differential forces are provided by user modulated means.

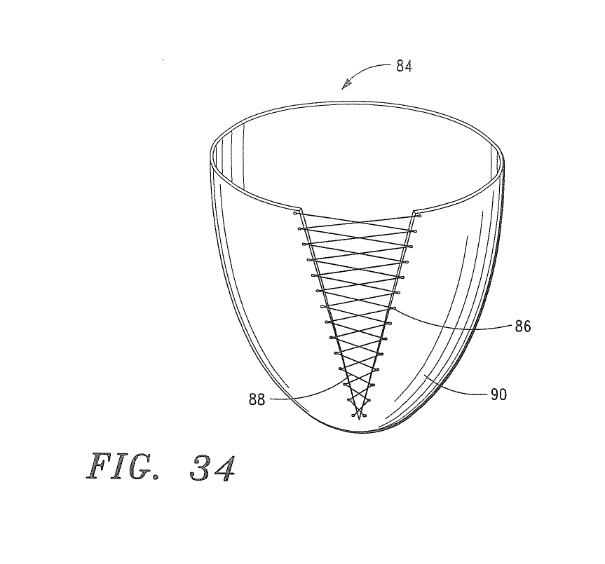

Referring now to FIG. 34, there is shown one embodiment of a biological construct comprising an organ encasement jacket that includes user modulated means for providing differential forces. As illustrated in FIG. 32, the organ encasement jacket 84 comprises a base jacket member 90 having a seam 86 configured to provide a user modulated differential force system by inserting and tightening sutures 88 disposed vertically along seam 86.

### EXAMPLES

The following examples are provided to enable those skilled in the art to more clearly understand and practice the present invention. They should not be considered as limiting the scope of the invention, but merely as being illustrated as representative thereof.

### Example 1

A biological construct comprising an ECM patch (i.e. matrix) comprising small intestine submucosa (SIS) and 1 mg/ml of a statin, i.e. cerivastatin, was surgically applied to the myocardium of two canines. The ECM patches remained attached to the myocardium of the canines until they were sacrificed at 2 and 24 hours, respectively.

Cardiac tissue samples were collected immediately after the canines were sacrificed. The cardiac tissue samples were then subjected to mRNA extraction and quantification via established protocols.

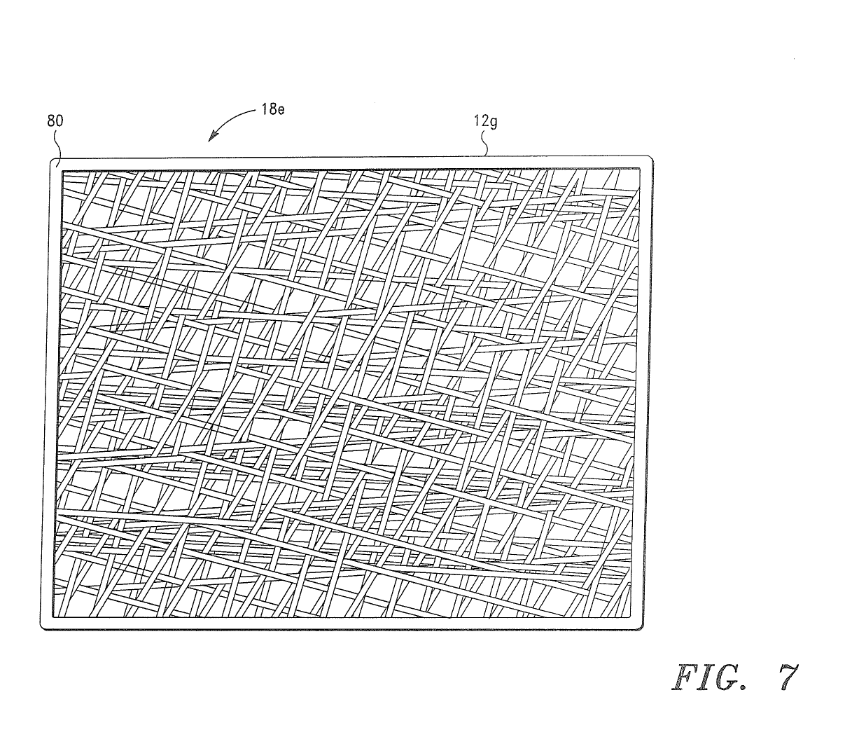

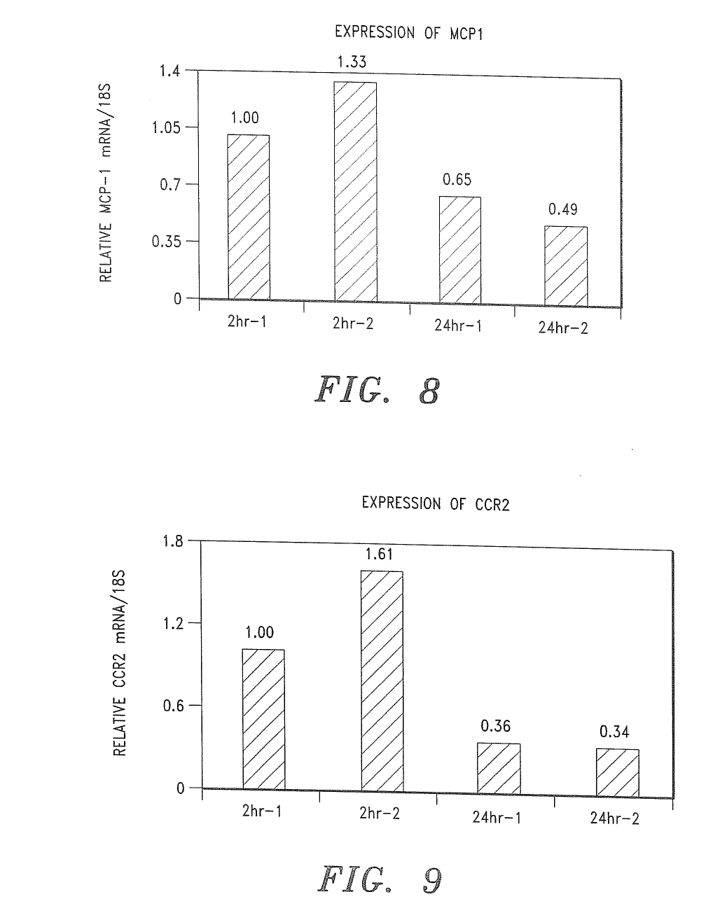

The measured mRNA levels from the cardiac tissue samples, which are shown in FIGS. 8-10, reflect substantially reduced MCP-1 and CCR2 expression at a 24 hour time point compared to the MCP-1 and CCR2 expression at a 2 hour time point. The mRNA levels thus reflect a consistent and highly effective anti-inflammatory effect over time in vivo, when a statin augmented ECM is administered to biological tissue.

The canine model experiment was further reinforced by an additional in vitro study, wherein MCP-1 expression of THP-1 cells (a human monocytic cell line) in the presence of a statin augmented ECM was analyzed. As reflected in FIG. 11, the statin augmented ECM induced substantially lower MCP-1 expression when compared to a positive control.

The example thus confirms that when a statin augmented ECM composition and, hence, a biological construct formed therefrom, is administered to damaged cardiovascular tissue, the biological construct will modulate several significant inflammation processes, including inhibiting generation of MCP-1 and CCR2.

The example further confirms that when a statin augmented ECM composition and, hence, a biological construct formed therefrom, is administered to damaged cardiovascular tissue, the biological construct will induce tissue proliferation and remodeling.

### Example 2

A sixty (60) year old male presents with a myocardial infarction characterized by an ischemic region on the wall of the left ventricle. Fibrotic scar tissue has also developed over the ischemic region of the left ventricle leading to abnormal wall motion (hypokinesia). As a result, the strength of left ventricular contraction is attenuated and inadequate for creating an adequate stroke volume and, hence, inadequate cardiac output, i.e. heart failure.

Prior to surgery, a reinforced biological construct comprising an organ encasement jacket is shaped and sized to be disposed over the right and left ventricles of the patient's heart based on an ultrasound 3-D model taken of the heart.

The reinforced organ encasement jacket comprises an ECM composition comprising small intestine submucosa (SIS) and TGF-β. The organ encasement jacket further comprises a first biologically active composition coating comprising SIS and a statin disposed on the encasement surface and a second biologically active composition coating comprising an ECM-mimicking composition comprising PGS disposed on the exterior surface.

The reinforced organ encasement jacket further comprises two linear manganese support members disposed on the exterior surface proximate the proximal end and mid-region of the jacket.

During surgery the patient is placed in a supine position and a median sternotomy is performed. An incision is made in the pericardium and the heart is temporarily suspended in cradle. The heart is gently lifted and the reinforced organ encasement jacket is stretched to fit over both the right and left ventricles of the heart; preferably, up to the pericardial reflection and over the entire ischemic region, whereby the reinforced organ encasement jacket provides a circumferential compressive force on both the left and right ventricles, which assists the heart during systole and, thereby, normalizing cardiac output.

Over the course of several weeks after the incision is closed, the reinforced encasement jacket induces modulated healing of the ischemic region and surrounding tissue, and adaptive regeneration of the left ventricle wall, resulting in a progressive normalization of the hemodynamic properties of the heart as the jacket biodegrades.

One having ordinary skill in the art will thus readily appreciate that the biological constructs of the invention provide numerous advantages over conventional apparatus and methods for supporting, treating damaged organs and/or associated tissue. Among the advantages are the following:
The provision of biological constructs that can be readily and effectively employed to treat damaged or diseased biological tissue; particularly, cardiovascular tissue;
The provision of biological constructs that can be readily employed to close and maintain closure of openings in biological tissue;
The provision of biological constructs that can be readily employed to support organ function;
The provision of biological constructs that induce “modulated healing” of damaged tissue, including host tissue proliferation, bioremodeling and regeneration of new tissue, and tissue structures with site-specific structural and functional properties, when disposed proximate the damaged tissue;
The provision of biological constructs that induce “adaptive regeneration and/or remodeling” of damaged organs and/or tissue associated therewith, including modulation of organ function, when disposed proximate thereto; and
The provision of biological constructs that effectively administer at least one biologically active agent and/or pharmacological agent or composition to a subject's tissue to induce a desired biological and/or therapeutic effect.

Without departing from the spirit and scope of this invention, one of ordinary skill can make various changes and modifications to the invention to adapt it to various usages and conditions. As such, these changes and modifications are properly, equitably, and intended to be, within the full range of equivalence of any issued claims.

What is claimed is:

1. An implantable biological construct for constraining and treating a damaged mammalian heart, comprising:
a jacket comprising a first extracellular matrix (ECM) composition comprising first ECM from a first mammalian tissue source,
said jacket further comprising an open proximal end, a distal end, an internal encasement surface and an exterior surface, said jacket defining an internal volume between said open proximal end and said distal end,
said jacket being configured to encase a region of said heart, wherein said jacket conforms to an external geometry of said heart region, and wherein, when said heart region includes a damaged tissue region and said jacket is positioned on said heart region, said jacket induces modulated healing of said damaged tissue region, said modulated healing comprising modulated inflammation and induced neovascularization and, thereby, remodeling of said damaged tissue region, and
wherein, during a cardiac cycle of said heart, said jacket concomitantly exerts a compressive force to said heart region, wherein said jacket induces adaptive regeneration of said heart, said adaptive remodeling comprising stress-induced hypertrophy.

2. The biological construct of claim 1, wherein, during said cardiac cycle of said heart, said jacket constrains circumferential expansion of said heart beyond a maximum adjusted volume during diastole.

3. The biological construct of claim 1, wherein said first mammalian tissue source is selected from the group consisting of the small intestine, large intestine, stomach, lung, liver, kidney, pancreas, placenta, amniotic membrane, heart, bladder, prostate and fetal tissue from a mammalian organ.

4. The biological construct of claim 1, wherein said first ECM composition further comprises a supplemental first biologically active agent.

5. The biological construct of claim 4, wherein said first biologically active agent comprises a first growth factor selected from the group consisting of transforming growth factor alpha (TGF-α), transforming growth factor beta (TGF-β), fibroblast growth factor-2 (FGF-2), vascular epithelial growth factor (VEGF), insulin-like growth factor (IGF) and hepatic growth factor (HGF).

6. The biological construct of claim 4, wherein said first biologically active agent comprises a first cell selected from the group consisting of embryonic stem cells, mesenchymal stem cells, hematopoietic stem cells, bone marrow stem cells, bone marrow-derived progenitor cells, myosatellite progenitor cells, totipotent stern cells, pluripotent stem cells, multipotent stem cells, oligopotent stem cells and unipotent stem cells.

7. The biological construct of claim 4, wherein said first biologically active agent comprises a first protein selected from the group consisting of collagen (types I-V), proteoglycans, glycosaminoglycans (GAGs), glycoproteins, cytokines, cell-surface associated proteins, and cell adhesion molecules (CAMs).

8. The biological construct of claim 1, wherein said first ECM composition further comprises a first pharmacological agent.

9. The biological construct of claim 8, wherein said first pharmacological agent comprises a first agent selected from the group consisting of an anti-viral agent, analgesic, antibiotic, anti-inflammatory, anti-neoplastic, anti-spasmodic, enzyme and enzyme inhibitor, anticoagulant, antithrombic agent and vasodilating agent.

10. The biological construct of claim 8, wherein said first pharmacological agent comprises a first HMG-CoA reductase inhibitor selected from the group consisting of atorvastatin, cerivastatin, fluvastatin, lovastatin, mevastatin, pitavastatin, pravastatin, rosuvastatin and simvastatin.

11. The biological construct of claim 1, wherein said jacket further comprises a coating disposed on said encasement surface, said coating comprising a biologically active composition.

12. The biological construct of claim 11, wherein said biologically active composition comprises a second ECM composition comprising second ECM from a second mammalian tissue source selected from the group consisting of the small intestine, large intestine, stomach, lung, liver, kidney, pancreas, placenta, amniotic membrane, heart, bladder, prostate, and fetal tissue from a mammalian organ.

13. The biological construct of claim 12, wherein said second ECM composition further comprises a supplemental second biologically active agent.

14. The biological construct of claim 13, wherein said second biologically active agent comprises a second growth factor selected from the group consisting of transforming growth factor alpha (TGF-α), transforming growth factor beta (TGF-β), fibroblast growth factor-2 (FGF-2), vascular epithelial growth factor (VEGF), and insulin-like growth factor (IGF).

15. The biological construct of claim 12, wherein said second ECM composition further comprises a second pharmacological agent.

16. The biological construct of claim 15, wherein said second pharmacological agent comprises a second agent selected from the group consisting of an anti-viral agent, analgesic, antibiotic, anti-inflammatory, anti-neoplastic, anti-spasmodic, enzyme and enzyme inhibitor, anticoagulant, antithrombic agent and vasodilating agent.

17. The biological construct of claim 15, wherein said second pharmacological agent comprises a second HMG-CoA reductase inhibitor selected from the group consisting of atorvastatin, cerivastatin, fluvastatin, lovastatin, mevastatin, pitavastatin, pravastatin, rosuvastatin and simvastatin.

18. The biological construct of claim 11, wherein said biologically active composition comprises an ECM-mimicking polymeric composition comprising poly(glycerol sebacate) (PGS).
